(19) United States
(12) Patent Application Publication
(10) Pub. No.: US 2014/0330320 A1
(43) Pub. Date: Nov. 6, 2014

(54) Bone Plate

(71) Applicant: Dietmar Wolter, Ahrensburg (DE)

(72) Inventor: Dietmar Wolter, Ahrensburg (DE)

(21) Appl. No.: 14/117,412

(22) PCT Filed: May 16, 2012
(86) PCT No.: PCT/EP12/59135

§ 371 Date: Jun. 16, 2014

(30) Foreign Application Priority Data
May 17, 2011 (EP) 11166395.1

(51) Int. Cl. A61B 17/80

(52) U.S. Cl. CPC A61B 17/8057
USPC 606/291; 606/280

(57) ABSTRACT

The application relates to a bone plate with a through-hole that extends from an upper face of the bone plate to a bone-side lower face thereof. A lip is formed in the through-hole, protrudes from the interior surface of the through-hole and extends in the circumferential direction of the through-hole. The lip has a plurality of circumferential portions in which the lip surface directed towards the upper face of the bone plate is designed as an inclined surface that slopes in the circumferential direction, wherein the lip surface has a front inclination face and a rear inclination face, and wherein the front inclination face and the rear inclination face have an opposite inclination in the circumferential direction. The bone plate has the advantage that a bone screw can easily cut into the lip.

### FIELD OF THE DISCLOSURE

The invention relates to a bone plate with a through-hole that extends from an upper face of the bone plate to a lower face thereof situated toward the bone. A lip formed in the through-hole protrudes from the lateral surface area of the through-hole and extends in the circumferential direction of the through-hole.

### BACKGROUND

Bone plates of this kind are designed to be connected to a bone. They can be used, for example, to stabilize a bone after a fracture. For this purpose, the bone plate is positioned such that it extends across the fracture site, and it is then secured on the bone fragments. The fracture site is thereby immobilized, and the bone is able to heal. The bone plate can also be used for other purposes and, for example, can be an element of an endoprosthesis to be connected to a bone.

To secure the bone plate on the bone, a bone screw is inserted into the through-hole, said bone screw being provided with a thread both on the shank and also on the head. The shank of the screw penetrates so far into the bone substance that the head of the screw enters the through-hole of the bone plate. The head of the bone screw is dimensioned such that its external diameter is greater than the smallest diameter of the lip. As the bone screw is screwed in further, the lip is deformed, such that a threaded connection forms between the head of the bone screw and the lip in the through-hole of the bone plate. Since there is a threaded connection both between the shank of the bone screw and the bone substance and also between the head of the screw and the bone plate, a secure connection is produced between the bone plate and the bone. Since the threaded connection between the head of the bone screw and the bone plate is formed only by a deformation process when the bone screw is screwed in, it is not necessary for the bone screw to be screwed at a specific predefined angle into the bone plate. Instead, the angle can be freely chosen within certain limits. This gives the surgeon a high level of flexibility during the operation.

In the previous lips, which extend uniformly in the circumferential direction of the through-hole, the thread on the head of the bone screw meets the lip at an acute angle. There is a danger of the thread first sliding along the surface of the lip before the deformation process begins and the thread engages in the lip. In this way, it is possible that the plate will move out of place before formation of the threaded connection, and this may lead to stress forces between the bone plate and the bone screw. Such stress forces may delay the healing of the bone.

### SUMMARY

The object of the invention is to make available a bone plate in which the risk of undesired forces acting on the bone is reduced. Proceeding from the background art set forth in the introduction, the object is achieved by the features of the independent claim. Advantageous embodiments are set forth in the dependent claims.

According to the invention, the lip has a plurality of circumferential portions in which the lip top surface directed toward the upper face of the bone plate is designed as an inclination face that slopes in the circumferential direction, wherein the lip top surface has a front inclination face and a rear inclination face, and wherein the front inclination face and the rear inclination face have an opposite inclination in the circumferential direction.

A number of terms will first be explained. A lip designates an area where the material thickness is reduced compared to the material thickness of the bone plate. The lip protrudes from the lateral surface area of the through-hole in the direction of the center of the through-hole. The lip can taper from the lateral surface area toward the center. Embodiments are also included in which this is not the case and in which, for example, the lip has the same material thickness near the center as it has near the lateral surface area. The lip has a longitudinal extent along the circumference of the through-hole. The lip has a lip top surface directed toward the upper face of the bone plate, and a lip underside directed toward the lower face of the bone plate, said top surface and underside each having an extent in the circumferential direction and an extent in the radial direction. The lower face of the bone plate lies on the bone when the bone plate is fitted.

If the lip top surface has an inclination face that slopes in the circumferential direction, a movement that follows the inclination face in the circumferential direction has an upward component in the direction of the upper face of the bone plate or a downward component in the direction of the lower face of the bone plate.

The lip preferably extends without interruption about the entire circumference of the through-hole. The inclination faces are then formed in portions of the lip. This means that the lip continues in the circumferential direction on both sides of the inclination face. In particular, both sides of the inclination face are adjoined by a portion of the lip in which the lip top surface has no slope in the circumferential direction.

The bone plate according to the invention has the advantage that the first contact takes place at a less acute angle when the head thread of the screw meets the inclination face. By virtue of the less acute angle, there is less risk of the thread sliding along the lip top surface instead of cutting into the lip and deforming the latter. The threaded connection between the bone screw and the bone plate is thus formed precisely in the position that the bone plate has upon the first engagement of the thread into the lip. Therefore, before the formation of the threaded connection, there is no shifting of the bone plate. Undesired stress forces between the bone plate, the bone screw and the bone are thereby avoided.

The lip comprises a front inclination face, and a rear inclination face with an opposite inclination in the circumferential direction. Front inclination face refers to an inclination face pointing in the direction of the thread of an associated bone screw. The first contact between the thread on the head of the bone screw and the lip will therefore take place on a front inclination face and not on a rear inclination face.

To increase the likelihood of the first contact between the head thread and the lip taking place on a front inclination face, the lip top surface can be provided with a plurality of front inclination faces. For example, the lip top surface can have at least three front inclination faces, preferably at least six front inclination faces. There is generally in each case one rear inclination face arranged between two front inclination faces, such that the number of the front inclination faces corresponds to the number of the rear inclination faces.

For the engagement of the head thread in the lip, it is advantageous if the head thread meets the front inclination face at an angle of the order of 90°. The likelihood of the head thread sliding along the lip top surface before the engagement takes place is then at its slightest. This can be achieved by the front inclination face having a larger gradient than the rear inclination face. Gradient designates the angle that the inclination face encloses with the horizontal in the circumferential direction. The gradient of the front inclination face can be between 60° and 90°, for example. The gradient of the rear inclination face can be between 20° and 60°, for example.

The likelihood of the first contact between the head thread and the lip taking place on the inclination face can be further increased if the inclination face has a large vertical extent. Vertical extent designates the distance from the lower face of the bone plate to the upper face of the bone plate. The inclination face preferably has a vertical extent of at least 30%, more preferably at least 50%, more preferably at least 70% of the thickness of the bone plate.

The lip underside can be plane. In alternative embodiments, the lip underside can likewise be provided with inclination faces. The number of the inclination faces on the lip underside can correspond to the number of the inclination faces on the lip top surface. Preferably, an inclination face on the lip underside is in each case substantially parallel to an inclination face on the lip top surface. This has the advantage that, at the first engagement, the head thread only has to displace a small amount of material of the lip before the head thread emerges again from the lip. In this way, a lip can be produced whose thickness is substantially constant in the circumferential direction and which extends as it were in the shape of a wave about the circumference of the through-hole.

The bone plate with the lip in the through-hole can be designed in one piece. Alternatively, it is possible that an area surrounding the through-hole is inserted in the form of an inlay into the bone plate. The inlay with the lip can firstly be formed as a separate part and can then be connected to the bone plate. To make the threaded connection easier to form, the inlay can be made from a softer material than the bone plate.

To allow the surgeon the option of using conventional bone screws in which the head is not provided with a thread, the lateral surface area of the through-hole can be widened outward above the lip. This part of the lateral surface area can then form an abutment surface for a conventional screw head. In cross section, the widening can have the shape of a segment of a circle, for example.

The invention also relates to a system composed of such a bone plate and of a bone screw. The bone screw has a head thread which is designed to deform the lip in the through-hole, in order to form a threaded connection. To permit secure fastening to the bone, the bone plate generally has a plurality of through-holes. Correspondingly, the system can also comprise a plurality of bone screws.

The material deformation taking place during the formation of the threaded connection should cause the least possible amount of chip abrasion. Tests have shown that the chip abrasion is kept low if the screw head has a conical lateral surface area and the angle that the lateral surface area encloses with the axis of the screw is between 19° and 28°.

### BRIEF DESCRIPTION OF THE DRAWINGS

The invention is described below by way of example on the basis of advantageous embodiments and with reference to the attached drawings, in which:

FIG. 1 shows a view of a bone plate according to the invention from above;

FIG. 2 shows a cross section through FIG. 1 along the line I-I;

FIG. 3 shows an enlarged view of detail A from FIG. 1;

FIG. 4 shows a view in the direction of arrow B in FIG. 3, with the cylindrical lateral surface area of the through-hole projected into the plane;

FIG. 5 shows the view from FIG. 4 in another embodiment of the invention;

FIG. 6 shows the view from FIG. 4 in a further embodiment of the invention;

FIG. 7 shows the view from FIG. 2 in another embodiment of the invention;

FIG. 8 shows a bone screw intended for the bone plate according to the invention; and

FIG. 9 shows the view from FIG. 2 in a further embodiment of the invention.

### DETAILED DESCRIPTION

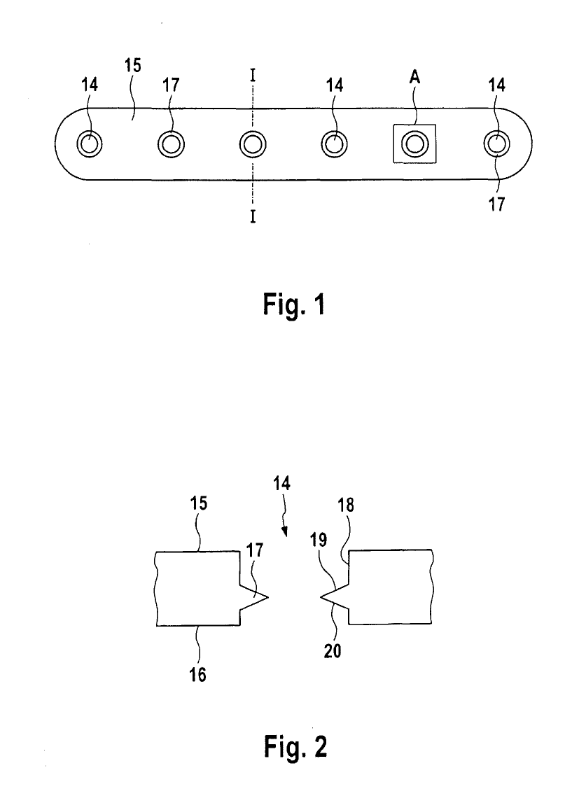

In accordance with FIGS. 1 and 2, a bone plate according to the invention has six through-holes 14 that extend from an upper face 15 of the bone plate to a lower face 16 thereof situated toward the bone. A lip 17 is formed in each through-hole 14, which lip 17 protrudes from the lateral surface area 18 in the direction of the center of the through-hole 14. The lip 17 is a continuous lip, which extends without interruption about the entire circumference of the through-hole. The lip 17 has a lip top surface 19 directed toward the upper face 15 of the bone plate, and a lip underside 20 directed toward the lower face 16 of the bone plate.

The bone plate is intended to be placed with the lower face 16 onto a bone, such that the bone plate extends across a fracture site of the bone. After the bone has been returned to the correct position, the bone plate is connected to the bone fragments. The bone is then fixed in this position and is able to heal.

To connect the bone plate to the bone, bone screws 31 are used, as are shown in FIG. 8. The bone screws 31 have a shank 27, which is provided with a bone thread 28, and a screw head 29 whose outer surface has a head thread 30 of smaller pitch. The lateral surface area of the screw head 29 encloses an angle of 25° with the axis of the screw. The bone screw is screwed into the bone until the screw head 29 enters the through-hole 14. The angle that the bone screw encloses with the axis of the through-hole can be freely chosen between about 0° and 15°. When the bone screw is now screwed in further, the head thread 30 of the bone screw 31 comes into engagement with the lip 17. The lip 17 is deformed, and a threaded connection is formed between the head 29 of the bone screw 31 and the lip 17. To make formation of the threaded connection easier, the bone screw 31 can be made from a harder material than the bone plate. For example, the bone plate can be produced from pure titanium, while the bone screw 31 is made from the titanium alloy TiAl6V4.

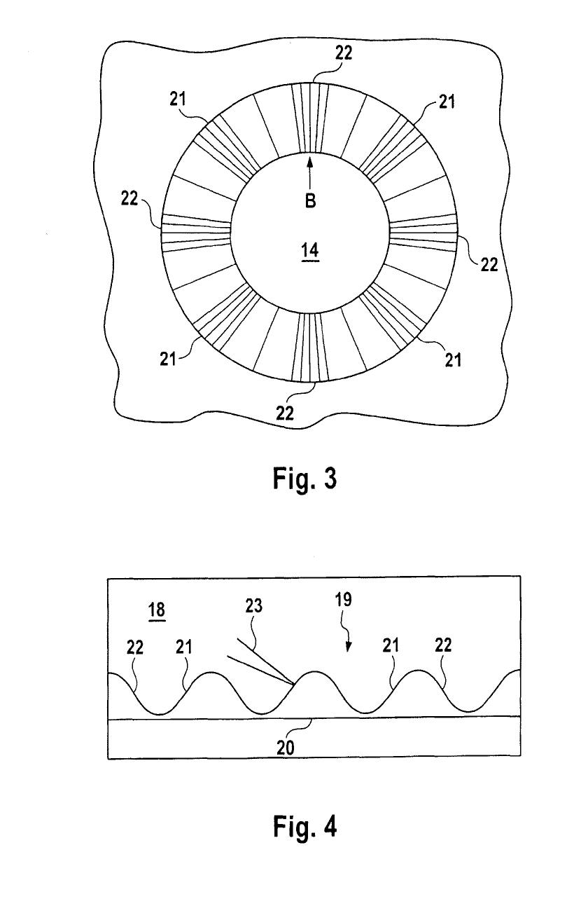

As is indicated by radial lines in FIG. 3, the lip 17 is provided, on the lip top surface 19, with a wave-shaped structure. In FIG. 3, the areas where the lip top surface 19 has a large gradient in the circumferential direction are identified by lines lying closely adjacent to each other. If one therefore moves in the circumferential direction on the lip top surface 19 transversely with respect to the closely adjacent lines, this is a movement in which one moves away from or approaches the upper face 15 of the bone plate. Thus, the lines lying closely adjacent to each other indicate the inclination faces on the lip top surface 19.

FIG. 3 shows a lip top surface 19 with four front inclination faces 21 and four rear inclination faces 22. If one moves clockwise in FIG. 3 along a front inclination face 21, one thus moves upward in the direction of the upper face 15 of the bone plate. The slope of the rear inclination faces 22 is the reverse. Therefore, if one moves clockwise in FIG. 3 along a rear inclination face 22, one moves downward and away from the upper face 15 of the bone plate. Between the front inclination faces 21 and the rear inclination faces 22, there is in each case an area in which the lip top surface 19 has no slope, i.e. it is oriented parallel to the horizontal.

While the bone screw is being screwed in, the first thread turn of the head thread comes into contact with the lip 17 at a certain point in time. Since the thread turn approaches the lip 17 obliquely from above, as is indicated at 23 in FIG. 4, it is highly likely that the first contact between the head thread and the lip 17 takes place in the area of a front inclination face 21. The thread turn meets the front inclination face 21 substantially at right angles, such that the thread turn can cut immediately into the lip 17 and deforms the latter. When, after further screwing, the conical screw head engages deeper in the lip 17, more material is displaced, and the screw head also enters the area where the lip 17 has a greater material thickness (see FIG. 2). A threaded connection forms that extends about the entire circumference of the screw head.

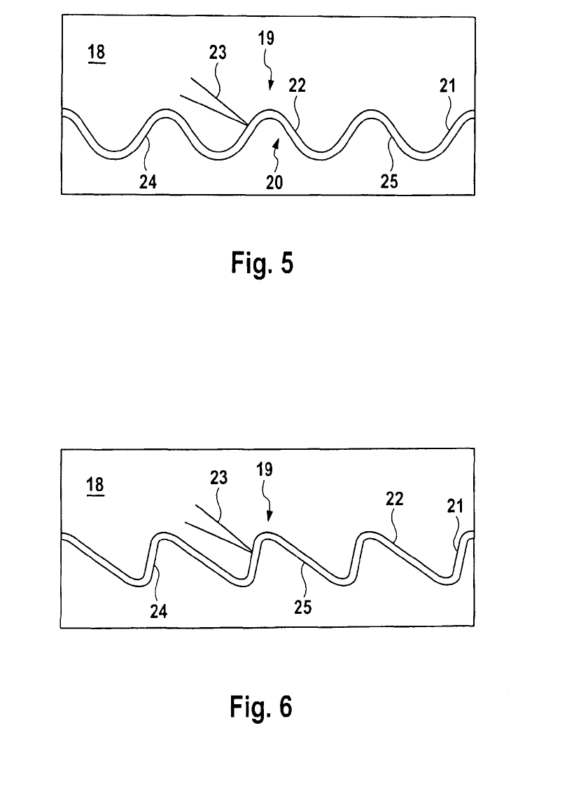

In the embodiment in FIG. 4, the underside of the lip is plane. In the alternative embodiment in FIG. 5, the underside 20 of the lip also has inclination faces 24, 25, which are each oriented parallel to the inclination faces 21, 22 on the lip top surface 19. After the first contact, the first thread turn, indicated at 23, of the head thread then has to displace only a small amount of material before it emerges again from the lip. For the surgeon, this has the advantage that no great resistance has to be overcome when screwing the head thread into the lip 17.

FIG. 6 shows a variant in which the front inclination faces 21 have a greater gradient than the rear inclination faces 22. The gradient of the front inclination faces is about 80°, and the gradient of the rear inclination faces is about 30°. This has the advantage that head threads with a small thread pitch also meet the front inclination face 21 substantially at right angles.

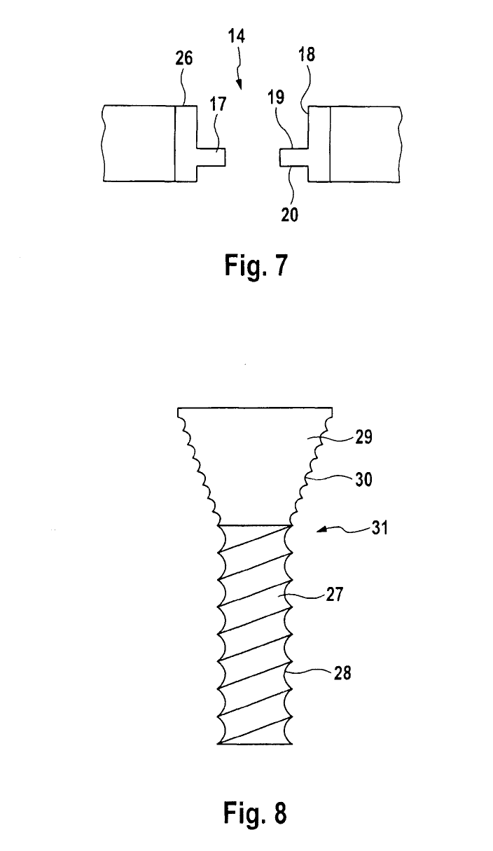

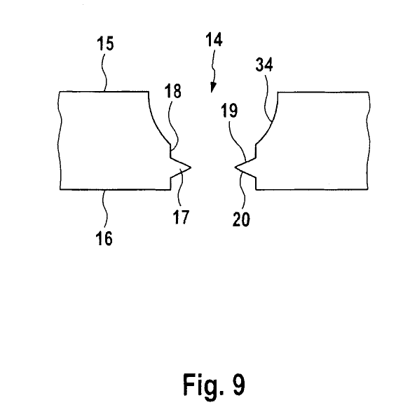

FIG. 7 shows an embodiment in which an inlay 26 is inserted into the bone plate. The inlay 26 is made from a softer material than the bone plate and forms the lip 17 and the surround of the through-hole 14. In contrast to FIG. 2, the lip 17 does not taper toward the center of the through-hole 14, and instead the lip 17 has a constant material thickness. In FIG. 9, the lateral surface area 18 above the lip 17 has a widening 34 which, in cross section, has the shape of a segment of a circle. With this embodiment, there is the option of using conventional bone screws in which the head does not have a thread. The widening 34 then forms an abutment face for a bone screw with a hemispherical head, such that the bone screw can be inserted at different angles into the through-hole 14.

1-10. (canceled)

11. A bone plate for fixture to a bone or bone fragment, said bone plate having an upper face and an opposed lower face oriented toward the bone and comprising a through-hole that extends between said upper and lower faces, said through hole having an inside surface and a lip formed in the through hole and projecting inward from said inside surface, said lip extending around a circumference of said through hole and having a top surface including a plurality of inclination faces that slope in the circumferential direction, said inclination faces including a plurality of front inclination faces inclined toward said upper face and a plurality of circumferentially adjacent rear inclination faces inclined away from said upper face.

12. The bone plate of claim 11, wherein said plurality of inclination faces includes at least three front inclination surfaces.

13. The bone plate of claim 11, wherein said plurality of inclination faces includes at least six front inclination surfaces.

14. The bone plate of claim 11, wherein each of said front inclination and rear inclination faces has an angle of inclination relative to a horizontal plane perpendicular to an axis of said through-hole and at least one of said front inclination faces has an angle of inclination greater than an angle of inclination of at least one of said rear inclination faces.

15. The bone plate of claim 14, wherein the angle of inclination of said at least one of said front inclination faces is between 60° and 90° and the angle of inclination of said at least one of said rear inclination surfaces is between 20° and 60°.

16. The bone plate of claim 11, wherein each of said front and rear inclination faces has an angle of inclination relative to a horizontal plane perpendicular to an axis of said through-hole and the angle of inclination of said front and rear inclination faces are substantially equal.

17. The bone plate of claim 11, wherein said inclination faces have a vertical extent at least 30% of a thickness of said bone plate.

18. The bone plate of claim 11, wherein said inclination faces have a vertical extent at least 50% of a thickness of said bone plate.

19. The bone plate of claim 11, wherein said inclination faces have a vertical extent at least 70% of a thickness of said bone plate.

20. The bone plate of claim 11, wherein said lip includes a substantially planar bottom surface.

21. The bone plate of claim 11, wherein said lip includes a bottom surface having a plurality of inclination faces parallel with corresponding inclination faces of said lip top surface.

22. The bone plate of claim 11, wherein an area surrounding said through-hole is inserted in the form of an inlay into said bone plate, said inlay defining said through hole.

23. A bone stabilization system comprising the bone plate of claim 11 and a bone screw having a shank and a head with an outside surface with a diameter larger than an inside diameter of said lip, said outside surface including a head thread which deforms said lip as said screw is threaded through said bone plate into a bone fragment.

24. The bone stabilization system of claim 23, wherein said shank includes a bone thread having a first pitch and said head thread having a second pitch smaller than said first pitch.

25. A bone stabilization system comprising the bone plate of claim 11 and a bone screw having a shank and a head with a conical outside surface, said conical outside surface subtending an angle of between 19° and 28° centered on an axis of said screw.

26. A bone plate for fixture to a bone or bone fragment, said bone plate having an upper face and an opposed lower face oriented toward the bone and comprising a through-hole that extends between said upper and lower faces, said through hole having an inside surface and a lip projecting inward from said inside surface, said lip extending around a circumference of said through hole and having a convoluted top surface including a plurality of front inclination faces inclined toward said upper face and a plurality of circumferentially adjacent rear inclination faces inclined away from said upper face.

27. The bone plate of claim 26, wherein said lip projects from said inside surface at a position closer to said lower face than to said upper face.

28. The bone plate of claim 26, wherein a thickness of said lip tapers from a root at said inside surface toward the center of the through hole.

29. The bone plate of claim 26, wherein said lip comprises a bottom face that is a mirror image of said top face.

30. The bone plate of claim 26, wherein said through hole has a first diameter at said upper face and a second diameter where said lip projects from said inside surface, said first diameter being greater than said second diameter.

31. The bone plate of claim 26, wherein said through hole is defined by an inlay that is inserted into the bone plate, said inlay is constructed of a material softer than the material of said bone plate.
